(19) United States
(12) Patent Application Publication
(10) Pub. No.: US 2015/0196251 A1
(43) Pub. Date: Jul. 16, 2015

(54) SYSTEM AND METHOD FOR MONITORING HUMAN WATER LOSS THROUGH EXPIRATION AND PERSPIRATION

(71) Applicants: Christopher Scott Outwater, Santa Barbara, CA (US); William Gibbens Redmann, Glendale, CA (US)

(72) Inventors: Christopher Scott Outwater, Santa Barbara, CA (US); William Gibbens Redmann, Glendale, CA (US)

(21) Appl. No.: 14/329,921

(22) Filed: Aug. 5, 2014

Related U.S. Application Data
(60) Provisional application No. 61/927,184, filed on Jan. 14, 2014.
(60) Provisional application No. 61/946,542, filed on Feb. 28, 2014.

(51) Int. Cl. A61B 5/00; A61B 5/11; A61B 5/1455; A61B 5/0205

(52) U.S. Cl. CPC A61B 5/4875; A61B 5/7278; A61B 5/742; A61B 5/7246; A61B 5/02055; A61B 5/1118; A61B 5/14551; A61B 5/4266; A61B 5/486; A61B 5/681; A61B 5/082

(57) ABSTRACT

A method and apparatus for determining and reporting water loss by a subject through expiration and perspiration. The reported water loss can be for a particular period of exercise or activity performance. The temperature and humidity of ambient air, e.g., as inhaled into the subject's lungs, and other factors including the temperature and humidity of the air exhaled, breath volume, and respiration rate, each of which may be measured, though some can be estimated on the basis of heart rate, exertion level, or recognized activities. Perspiration rate can also be measured and included. Monitoring for pulmonary hemorrhage or hemoptysis can also be incorporated.

This application claims priority under 35 U.S.C. 119(e) to 1) U.S. Provisional Application No. 61/927,184 entitled “SYSTEM AND METHOD FOR TESTING AIR EXHALED FROM LUNGS”, filed Jan. 14, 2014; and, 2) U.S. Provisional Application 61/946,542, entitled “SYSTEM AND METHOD FOR MONITORING LOSS OF FLUIDS THROUGH EXPIRATION AND TRANSPIRATION” [sic], filed Feb. 28, 2014; incorporated by reference in their entireties.

### FIELD OF THE INVENTION

The invention relates generally to a system and method for measuring human or animal hydration loss with a sensor, and more particularly determining water loss through respiration by measurement and estimation.

### BACKGROUND

A normal hydration level is key to normal bodily function and is especially important for infants, athletes, and the elderly. These groups often do not correctly interpret bodily signs that indicate a need for more hydration. Low hydration in athletes can lead to poor performance and muscle cramping and in the elderly can lead to many problems, including high blood pressure and stroke.

The problem is that there is presently no method to easily and quickly measuring body hydration levels. The two most reliable ways for measuring hydration today involve testing either of a subject's urine or blood. Another way is to measure the patient's blood pressure while lying down and then again while standing up. Each of these tests take time, cost money, and are often otherwise inconvenient. In particular, blood tests may expose the patient to potential accidents and infection. Testing skin turgor (with the “pinch test”) is another method, observing how fast a pinched fold in abdomen or thigh relaxes, but the pinch test is very subjective and not suitable for obese individuals.

Water lost in urine is more easily perceived than water lost by other paths. The corresponding volume or weight of replacement water is essentially the same as the volume or weight of the urine itself. Further, in athletic situations, frequency of urination is generally lower and thus of less consequence.

Water lost through perspiration is more difficult to perceive, especially if the environment is not humid and perspiration evaporates quickly leaving little trace and correspondingly little opportunity to notice it.

Water lost through respiration is still more difficult to perceive, since the form of the loss is water vapor, and is invisible, except on particularly cold days.

### OBJECTS AND SUMMARY OF THE INVENTION

“Exhalant” is exhaled air, also called expired air. Similarly, “inhalant” is inhaled air. The present invention is an exhalant monitor to determine the amount of water loss through respiration based on breath volume, aggregate number of breaths, and the difference between the humidity of air inhaled and that of the air exhaled. Breath volume and the number of breaths may each be measured or estimated based on exertion and/or activity. In some embodiments, a further amount of water loss by perpiration through the skin is determined from the measurements of the ambient air and a measurement or estimate of at least one of perspiration and exertion. Some embodiments further monitor exhalant for the presence of red blood cells, to detect for exercise-induced, high altitude-induced, or trauma-induced pulmonary hemorrhage, or more serious conditions.

It is an object of the present invention to determine water loss through respiration.

It is a further object of the present invention to additionally determine water loss through perspiration.

It is an objection of the present invention to advise an amount of replacement water needed based on water lost during a period of exercise.

Another object of the present invention is to estimate breathing rate robustly, so as to continue estimation when a primary breathing rate measurement is disrupted.

Various devices suitable to monitor water loss through expiration, and operations thereof as described herein, with the further addition of an appropriate sensor to measure red blood cells in the exhalant, are also sufficient to monitor for, recognize, and provide an early warning of potential pulmonary hemorrhage (bleeding into the lungs, as might be induced by exercise, especially at high-altitude, or due to trauma).

Accordingly, it is an object of the present invention to monitor a subject's exhalant to detect and warn of pulmonary hemorrhage: It is an object of the present invention to monitor for and warn of blood cells in a subject's exhalant.

Embodiments of the present invention relate to a method and system for monitoring water loss by a subject, by automatically determining an amount of water loss based on at least the humidity of the environment and a plurality of proxy values read from a sensor which are interpreted by a controller with a subject profile to provide at least one of a breathing rate and a breath volume.

One embodiment provides a system for monitoring water loss which includes a humidity sensor, an air flow sensor inside a test chamber, at least one other sensor that provides a proxy value related to breathing rate or breath volume of a subject, a controller having communication with the sensors and a memory, wherein, with the sensors, the controller can determine the humidity of the environment, a breathing rate of the subject, a breath volume of the subject when the subject breathes through the test chamber, and can create a profile in memory by which the proxy value is related to at least one of the breath volume and the breathing rate of the subject, from which the controller can determine and report an amount of water loss over an interval based on a plurality of proxy values based on the humidity, the profile, and readings of the at least one other sensor during the interval.

Another embodiment provides a controller-implemented method for use in monitoring water loss which includes the steps of accepting the humidity of the environment, reading a profile from memory that associates a proxy value with at least one of a breath volume and breathing rate, determining a plurality of proxy values for a subject based on readings over an interval of at least one sensor (e.g., a heart rate monitor, a perspiration sensor, a motion sensor), and from the plurality of proxy values, the humidity, and the profile, determining and reporting an amount of water loss by the subject over the interval.

### BRIEF DESCRIPTION OF THE DRAWINGS

The aspects of the present invention will be apparent upon consideration of the following detailed description taken in conjunction with the accompanying drawings, in which like referenced characters refer to like parts throughout, and in which:

FIG. 1 shows two phases of respiration, illustrating parameters of water loss;

FIG. 2 shows a respiration water loss monitor in an example wrist-mounted embodiment with one example block diagram;

FIG. 3 shows a respiration water loss monitor in an example smartphone-based embodiment with another example block diagram;

FIG. 4 shows a flowchart for one example respiration water loss monitoring process;

FIG. 5 shows a flowchart for estimating breathing rate; and,

FIG. 6 shows one example configuration for an airborne blood cell sensor.

While the invention will be described and disclosed in connection with certain preferred embodiments and procedures, it is not intended to limit the invention to those specific embodiments. Rather it is intended to cover all such alternative embodiments and modifications as fall within the spirit and scope of the invention.

### DETAILED DESCRIPTION

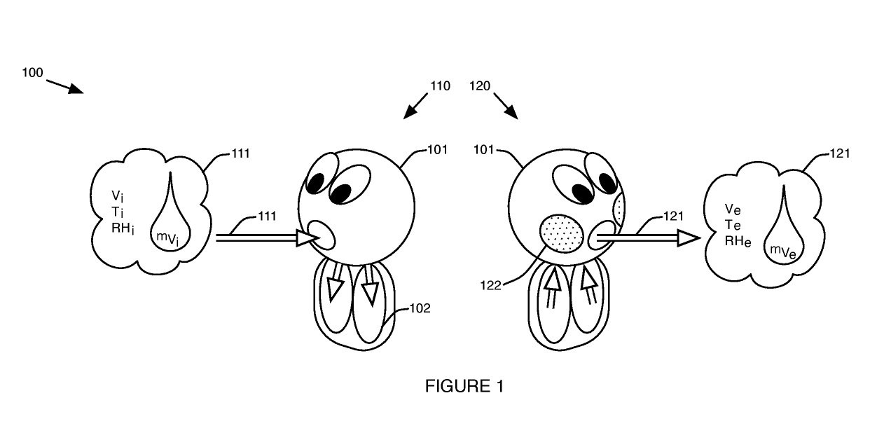

Water loss by respiration is illustrated in FIG. 1 for breathing cycle 100, where the lungs 102 of subject 101 fill (though not necessarily entirely) during inhaling phase 110, and subsequently empty (again, not necessarily entirely) during exhaling phase 120. The inhalant 111 is the air taken in by subject 101 during inhaling phase 110 and exhalant 121 is the air released by subject 101 during exhaling phase 120. In some cases, the cheeks 122 of subject 101 puff while exhaling, particularly if the subject is breathing hard. For clarity, puffed cheeks 122 are used herein merely to illustrate that the subject 101 is exhaling.

Inhalant 111 is characterized by a volume (V_{INHALED}), a temperature (T_{INHALED}), a humidity (in FIG. 1 expressed as relative humidity RH_{INHALED}), which collectively correspond to a first mass of water vapor (m_{Vinhaled}). Likewise, exhalant 121 is characterized by a volume (V_{EXHALED}) a temperature (T_{EXHALED}), a humidity (in FIG. 1 expressed as relative humidity RH_{EXHALED}), which collectively correspond to a second mass of water vapor (m_{Vexhaled}). The net water loss in breathing cycle 100 for the breath taken and released during inhaling phase 110 and exhaling phase 120, respectively, is the difference between the second and first masses of water vapor, or m_{Vexhaled}−m_{Vinhaled}.

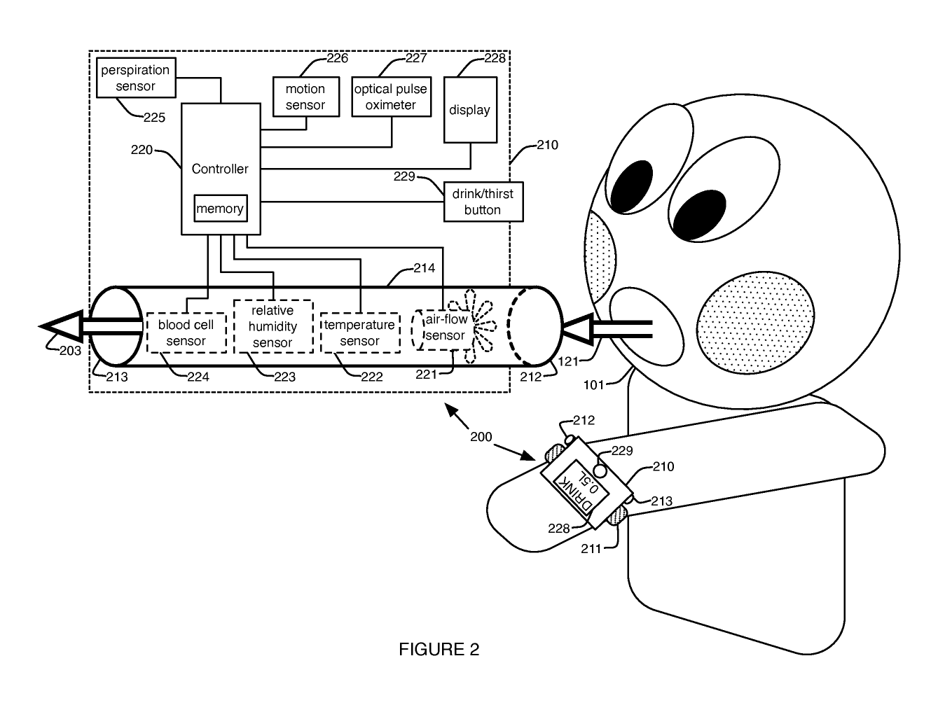

FIG. 2 shows one example wrist-mounted embodiment for a water loss monitor 200. On the wrist of subject 101, monitor 200 is shown comprising housing 210, wristband 211 for holding the monitor in position, mouth port 212 to receive exhalant from subject 101, ambient port 213 through which exhalant leaves the monitor 200 as exhaust 203, and display 228 on which the monitor presents information to subject 101. In this embodiment, a button 229 represents one or more buttons or touch screen elements to serve as a user interface for subject 101 to signal thirst, and/or to signal that the recommended amount of water has been consumed. Button 229 can also allow the user to signal the beginning and/or end of a particular activity or period of exercise; or, such activities or periods can be recognized automatically (as discussed further, below) Thus, by way of illustration and not limitation, water loss monitor 200 is self-contained.

FIG. 2 also shows an enlarged view of example water loss monitor 200, including a block diagram, in use during an exhalation phase 120. Subject 101 releases a breath as exhalant 121 into mouth port 212. Mouth port 212 may feature a mouthpiece (not shown) or may merely be a tube or straw for subject 101 to blow into. Subject 101 should make a good seal (not shown) with mouth port 212 to ensure that the whole of exhalant 121 enters port 212. Mouth port 212 and ambient port 213 are openings into test chamber 214, here shown by way of example as a tube. Test chamber 214 contains sensors 221-224, each readable by controller 220. When monitor 200 is being actively used to measure exhalant 121, the nose of subject 101 will typically be held (e.g., with the hand opposite that bearing wrist-mounted device 200) to as to prevent loss of exhaled air other than through mouth port 212.

Airflow sensor 221, in this embodiment shown as a propeller-based anemometer, is used to determine the volume V_{EXHALED} of the exhalant 121 by measuring the air velocity, multiplying by the cross-sectional area of the chamber 214 in proximity to sensor 221 and integrating over the duration of the exhalation (which would be from when airflow from exhalant 121 begins until it ceases). Controller 220 comprises or otherwise has communication with a clock (not shown), which enables measurement of such intervals of time. Subject 101 could also inhale through mouth port 212, in which case ambient air would flow in through ambient port 213 and the air velocity measured by airflow sensor 221 would reverse direction and a similar integration would provide the volume V_{INHALED} of the inhalant 111 from inhalation phase 110 (not shown in FIG. 2).

Temperature sensor 222 and humidity sensor 223 (here a relative humidity sensor) measures the temperature T_{EXHALED} and, in this embodiment, relative humidity RH_{EXHALED} of exhalant 121. Relative humidity is “the ratio of the partial pressure of water vapor to the saturated vapor pressure of water, at a given temperature”. As will be discussed in more detail below, given the relative humidity, a corresponding temperature is also required to determine the density (mass per unit volume) of water vapor in the air.

One such sensor is a capacitance-based humidity sensors, in which the relative static permittivity of a polymer or metal oxide dielectric varies according to the relative humidity. An example integrated temperature sensor 222 and humidity sensor 223, suitable for this embodiment, is the Si7020 by Silicon Labs, Inc. of Austin, Tex., which comprises a humidity sensor, temperature sensor, an analog-to-digital converter, control logic, and a calibration memory for converting signals from the humidity and temperature sensors into a relative humidity reading. The relative humidity and temperature readings are each accessible to an external controller (e.g., controller 220) through an I2C interface. An alternative choice for a relative humidity sensor is a resistive humidity sensor that relies on a hygroscopic medium whose resistivity varies with relative humidity.

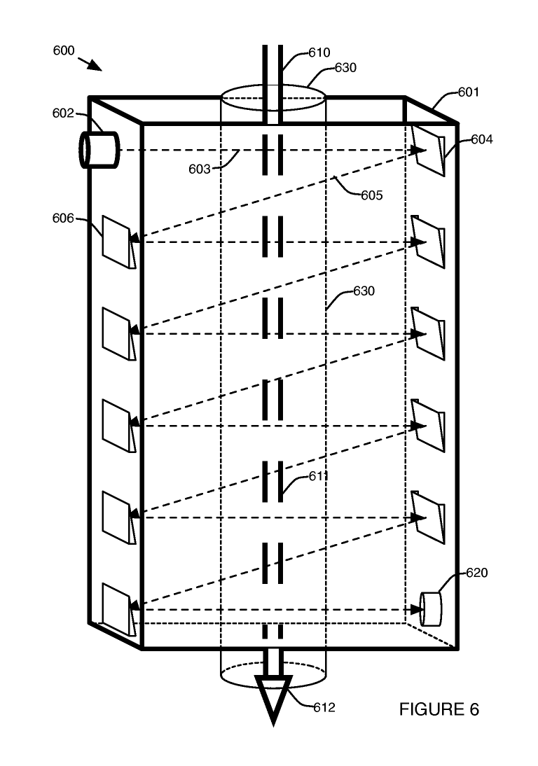

Blood cell sensor 224 detects the present of red blood cells in exhalant 121, and is discussed in greater detail below in conjunction with FIG. 6.

Over the duration of the exhalation, controller 220 receives the airflow, temperature, and humidity measurements corresponding to exhalant 121 and determines the corresponding volume (V_{EXHALED}), temperature (T_{EXHALED}), and relative humidity (RH_{EXHALED}).

Over a sufficiently long interval (e.g., several minutes) without the subject 101 releasing exhalant 121 into monitor 200, which is to say, not using the mouth port 212, chamber 214 will come into equilibrium with the environment and temperature sensor 222 and humidity sensor 223 will register the ambient temperature and relative humidity, which would correspond to temperature (T_{INHALED}) and relative humidity (RH_{INHALED}) of inhalant 111. An acceptable approximation is that the volume (V_{INHALED}) of the inhalant 111 (not shown in FIG. 2) corresponding to exhalant 121 is equal to the exhalant volume (V_{EXHALED}). Thus, given sufficient time to come to equilibrium with the environment, readings made immediately prior to the subject 101 releasing exhalant 121 into mouth port 212 can be used to characterize the corresponding inhalant 111.

Alternatively, during inhalation phase 110, subject 101 can draw inhalant 111 (not shown in FIG. 2) through mouth port 212, causing ambient air to be drawn in backwards through ambient port 213. In this case, temperature sensor 222 and relative humidity sensor 223 will characterize those parameters of the inhalant 111. In a calculation similar to that for volume V_{EXHALED}, measurements from airflow sensor 221 will be used to determine the volume V_{INHALED} of inhalant 111.

In another embodiment, the temperature and relative humidity of the ambient air can be measured with appropriate sensors (not shown) external to chamber 214 and connected to controller 220. In some embodiments, particularly if monitor 200 is being used outdoors, temperature and relative humidity could be supplied from current weather data.

So far, monitor 200 is shown to measure characteristics of inhalant 111 and exhalant 121. This is sufficient to determine an incremental loss of water for a single breathing cycle 100, for which calculations are shown below.

Outside of critical medical situations, subject 101 would likely object to drawing and releasing every breath through monitor 200. It would be significantly inconvenient. To address this, monitor 200 uses the determined water loss corresponding one breathing cycle (or several) as the basis for determining water loss over longer intervals and many breathing cycles, but to determine how many breaths are taken during a measurement interval and aggregate the total respiratory water loss when not breathing through monitor 200, the number of breaths must be counted or estimated in some other way, which the present invention provides.

While breaths can be counted using airflow sensor 221, because of the inconvenience cited above, alternative ways to count are useful for longer intervals, using a sensor other than the airflow sensor 221. For example, breaths can be counted with an acoustic monitor such as the Rainbow Acoustic Monitor by Masimo Americas, Irvine, Calif. (not shown); or by using an optical pulse oximeter sensor 227, as taught by Leonard, et al. in Standard pulse oximeters can be used to monitor respiratory rate, Emerg Med J 2003; 20:524-525. Other breath-counting mechanisms (not shown) are known and may be used instead of, or in addition to, these.

In monitor 200, optical pulse oximeter sensor 227 measures O₂ saturation, that is, the percentage of blood hemoglobin loaded with oxygen, by monitoring the differential absorption of different light wavelengths by blood through translucent skin, e.g., the wrist skin immediately beneath wrist-worn monitor 200, but the measured value varies with each heartbeat and, as Leonard points out, each respiration cycle. Decomposition by controller 220 of the pulse oximeter waveform from sensor 227 using wavelet transforms determines the breathing rate as the dominant reading in the frequency band below the heart rate, from which individual breaths are counted. As monitor 200 is wrist-worn and optical pulse oximeter sensor 227 is in direct contact with the wrist of subject 101, the ability to count breaths is non-invasive and on-going without explicit action taken by subject 101.

However, during periods of vigorous exercise or other disruptive movement, the signal from sensor 227 may become too noisy or otherwise unsuitable for effectively extracting a signal for reliably counting breaths. In such instances, the breathing rate is estimated.

For example, in one embodiment, the simplest way to estimate subsequent breathing rate is to merely consider that the most recently observed breathing rate continues, until such time as the signal from sensor 227 is again suitable for counting breaths. Thus, if the breathing rate was 15 breaths per minute before readings from sensor 227 were disrupted, controller 220 can estimate one breath for every four seconds (60 seconds/15 breaths per minute) until the readings are restored.

In another embodiment, a memory within, or otherwise available to, controller 220 is used to store data representing a profile for a subject, or “subject profile”. For this purpose, the subject profile data comprises records representing a correspondence between at least one proxy breathing rate parameter vs. expected breathing rates.

One choice for the proxy breathing rate parameter can be “activeness”, to be measured using motion sensor 226, for example, a three-axis accelerometer, such as the ADXL345 digital accelerometer from Analog Devices, Inc. of Norwood, Mass. Integrating the sum of the squares of the acceleration in each axis, measured frequently (e.g., many times per second) over long intervals (e.g., a minute). Considering that these acceleration readings represent movement of a particular portion of the body of subject 101 (the left wrist, in the case of wrist-mounted water loss monitor 200 worn as shown in FIG. 2). Readings obtained from the same monitor, similarly worn during similar activities by the same subject will likely be similar. Further, the subject's breathing rate during the same activity performed at a similar level of exertion is likely to be similar, one day to the next, especially when the activity is prolonged. Accordingly, “activeness” readings taken in conjunction with successful breathing rate measurements obtained from two or more consecutive successful breath counts and recorded in the memory (or merged with similar records in the memory), provide a basis for estimating a similar breathing rate the when similar “activeness” is detected.

Another choice for the proxy breathing rate parameter is a recognized activity. Systems are known that recognize a particular exercise (i.e., activity) based on particular patterns of motion, e.g., as measured by motion sensor 226 and/or other sensors, e.g., a global positioning system (GPS) sensor (not shown), for example as taught by Redmann in U.S. Pat. Nos. 8,109,858 and 8,343,012. Distinct activities to be recognized can include walking, walking quickly, jogging, running, climbing up stairs, climbing down stairs, playing basketball, playing soccer, etc. Records noting that certain activities were recognized, or that a certain count of a particular activity have been recognized, in conjunction with successful breathing rate measurements can be recorded in memory. As similar actions are recognized individually or accumulated as counts (e.g., 25, 50, or 75 jumping jacks), a breathing rate estimate can be determined based on records noting similar actions. Such automatic recognition of activities, or of a period of exercise, and their beginning and/or end, in lieu of the subject pressing button 229 as discussed above, can be the basis for the interval over which a water loss determination is made.

Still another choice for the proxy breathing rate parameter is heart rate. While varying with instant circumstances and between different individuals, heart rate (heartbeats per minute) and breathing rate (breaths per minute) maintain a ratio of roughly 4:1. Accordingly, a sensor able to determine heart rate, e.g., optical pulse oximeter sensor 227, can do so with an interval of readings about ¼th the duration necessary to determine breathing rate. If sensor disruption (e.g., by noise or intermittency) is too great for breathing rate to be detected, thereby inducing the need for a proxy breathing rate parameter, it may still be possible that the same sensor (or a different one) is still able to deliver measurements of sufficient quality for heart rate to be determined. The reason for this, besides heart rate being faster, is that, in the example of an optical pulse oximeter sensor, the signal resulting from each pulse of blood (systolic phase) and the relaxation interval (diastolic phase) that follows is considerably more prominent than the variations in those readings induced by respiration phase, such that the heart beat signal has about three to seven times the signal level of the breathing rate signal, and so a likewise advantaged signal-to-noise ratio. Thus, even when the signal generated by sensor 227 is usable for determining breathing rate directly, the signal can be adequate for determining heart rate, which in turn can be used as a proxy breathing rate parameter. As with the activeness- and activity-based proxy breathing rate parameters, records stored in the memory by controller 220 can represent correlations between measured breathing rate and heart rate as a proxy breathing rate parameter. When required, heart rate can be used with such records from the subject profile data to produce an estimated breathing rate.

As with breathing rate, except for critical medical situations, breath volume (V_{INHALED} and/or V_{EXHALED}) is likewise inconvenient to measure directly for every breath with airflow sensor 221. In ways similar to the above, breath volume can be estimated to be the same as a measured breath volume, either one or both of V_{INHALED} and V_{EXHALED}, or as an average of several measured breaths. In some embodiments, this is a calibration breath, measured to tune monitor 200 to subject 101 by storing the result as a further record in the subject profile data. In another embodiment, this could be a recently measured breath. In still another embodiment, this could be a subsequently measured breath, the measurement of which is retroactively applied to previously estimated or measured breath counts. Historically measured breath volumes may be stored in the memory of controller 220 for such use. In a manner similar to those discussed above, breath volume can be estimated based on a proxy breath volume parameter, which as discussed above may comprise activeness, a recognized activity, heart rate, breathing rate, or other measurable parameter. Records are made of actual measured breath volumes (whether V_{INHALED} or V_{EXHALED}) taken in substantial conjunction with (e.g., within a minute of) a determination of activeness, recognized activity, heart rate, breathing rate, etc. Given such a record, a measured (or estimated) value for the proxy breath volume parameter can be used to estimate a corresponding breath volume using the records.

Such estimates for breathing rate and breath volume might be estimated in certain ways, even when ways of actual measurement or more precise estimations are available, in order to save energy. For example, even in cases where the signal sensor 227 remains suitable for counting breaths, i.e., has not been disrupted by noise or interruption, controller 220 may be configured to discontinue actual measurement and power down sensor 227, perhaps for one to several minutes, to save energy and reduce the drain on a battery (not shown). During this interval, breathing rate and/or breath volume can be estimated to retain their previous values without too greatly departing from the quality of the more accurate counting. The power savings results both from non-operation of the optical pulse sensor 227 (a collection of LEDs, photo-detectors, amplifiers, and analog-to-digital converters that consume power when in use) and reduced power consumption by controller 220, since the wavelet analysis to extract breathing information is not performed, saving the corresponding computational effort, perhaps allowing the controller 220 to enter a reduced-power mode (e.g., a sleep mode) instead.

In some embodiments, perspiration sensor 225 is provided to measure perspiration by subject 101. Moisture emitted by the skin of subject 101 will subsequently evaporate, the translation of a measured perspiration value into a water loss value further requires a determination of the rate of evaporation, which is based on the humidity of the surrounding air, body temperature, air temperature, and even the velocity of the air surrounding the subject 101. Given air less than saturated with water vapor, the rate of evaporation will depend on the relative humidity and temperature. A suitable perspiration sensor 225 is taught by Salvo, et al. in A Wearable Sensor for Measuring Sweat Rate, IEEE Sensors Journal, Vol. 10, No. 10, p 1557-1558, October 2010.

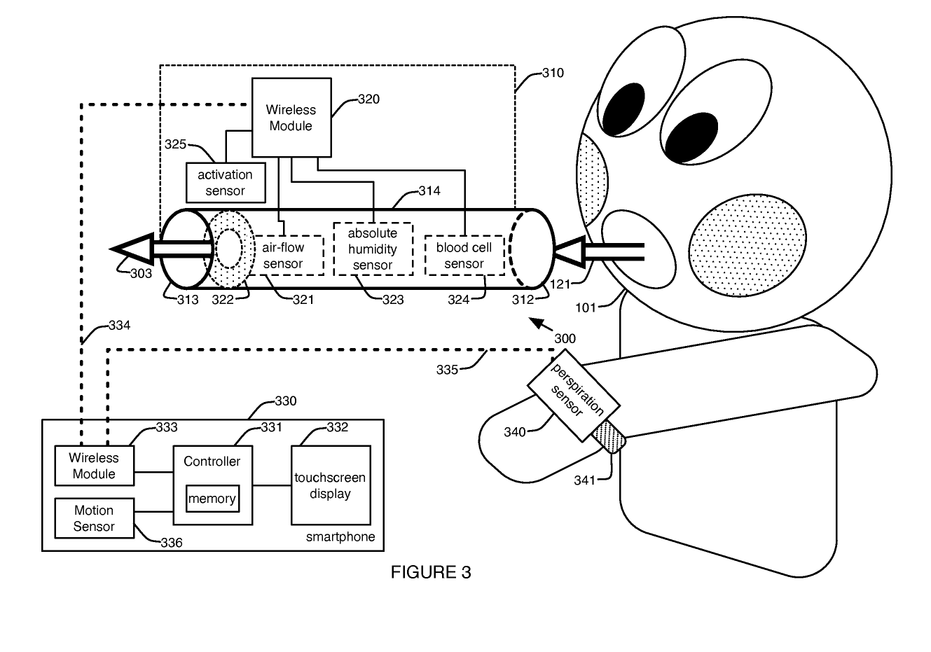

Another embodiment of the present invention is shown in FIG. 3, where water loss monitor 300 is not self-contained, but operates in conjunction with smartphone 330 through wireless connection 334. As above, subject 101 directs exhalant 121 through this mouth port 312 into test chamber 314. Exhalant exits test chamber 314 through ambient port 313 as exhaust 303. Test chamber 314 contains air-flow sensor 321, absolute humidity sensor 323, and blood cell sensor 324. Each of sensors 321, 323, and 324 provide readings to controller 331 of smart phone 332 by way of wireless connection 334 provided by wireless modules 320 and 333.

Another sensor introduced in FIG. 3, but independent of whether or not the embodiment is self-contained or not, is activation sensor 325, provided to detect that monitor 300 is about to be used. In one embodiment, activation sensor 325 could be a button to be pressed or otherwise activated by subject 101. In an alternative embodiment, sensor 325 could be an accelerometer detecting that monitor 300 has been moved from a first position (e.g., from a pocket or belt-clip, neither show) to a second position characteristic of being used (i.e., having a certain orientation relative to the Earth's gravitational field). In still another embodiment, activation sensor 325 could be a capacitive sensor connected to detect contact between a mouthpiece (not shown) at mouth port 312 and the mouth of subject 101. Activation sensor 325 can activate wireless module 320.

In some embodiments, wireless module 320 may comprise a controller (not shown) to manage readings of sensors 321, 323, 324. In some of such embodiments, wireless module 320 may be reactive to activation sensor 325, e.g., powering down when activation sensor 325 indicates the monitor 300 is not position for use and powering up when use is imminent. In other such cases, activation sensor 325 may provide a signal through wireless module 320 to controller 331. One particular use of the signal produced by activation sensor 325 is that can coincide with a moment where sensor readings for temperature and/or humidity (e.g., from sensor 323, (or in another embodiment, sensors 222, 223) would correspond to ambient values.

Motion sensor 336 and touchscreen display 332 of smartphone 330 correspond in function and operation to motion sensor 226 and display 228 in the embodiment of monitor 200. Further, touchscreen display 332 can also serve as button(s) 229, as discussed above. Controller 331 comprises or otherwise has communication with a clock (not shown), suitable for measuring time intervals as discussed in conjunction with FIG. 2. A separate perspiration sensor 340, wrist mounted to subject 101 by wristband 341 substitutes for perspiration sensor 225, though perspiration sensor 340 communicates with controller 331 by wireless connection 335.

Another feature of FIG. 3, differing from FIG. 2, and also independent of whether monitor 300 is self-contained or not, is air-flow sensor 321, which differs from air-flow sensor 221: Air-flow sensor 321 here is pressure-based and determines the rate of air-flow according to Bernoulli's principle, by measuring the rise above ambient atmospheric pressure as the subject 101 exhales into the mouth port 312 and the exhalant 121 flows past sensor 321 and through the rest of test chamber 314. In some embodiments, a calibrated orifice plate 322 can be provided to dominate the fluid mechanics of air-flow through chamber 314 and to magnify the differential pressure between the ambient air pressure and the exhalant entering chamber 314. Measurements from pressure sensing air-flow sensor 321 made before or after, but not while, subject 101 breathes through monitor 300 represent the ambient atmospheric pressure, which when used in conjunction with a measurement made while subject 101 is exhaling (or inhaling) through monitor 300, will provide a pressure differential. In conjunction with the temperature of the exhalant and certain factors about humid air, the volume of exhalant can be determined. For example:

$$Δ {V}_{EXHALED} = CA \sqrt{\frac{2 {ZRT}_{EXHALED}}{{M}_{EXHALED}} ( \frac{k}{k - 1} ) [ \begin{matrix}{( {P}_{AMBIENT} / {P}_{EXHALED} )}^{2 / k} - \\ {( {P}_{AMBIENT} / {P}_{EXHALED} )}^{k + 1 / k}\end{matrix} ]} \qquad EQ . 1$$

Where ΔV_{EXHALED} is the gas flow rate of the exhalant (or inhalant) in units of meters³/second, ‘C’ is the flow coefficient, which is a dimensionless characteristic of the overall flow path for air through test chamber 314 (including ambient port 313), but is substantially governed by orifice plate 322, especially where the cross-sectional area ‘A’ (in meters²) of the orifice plate 322 is significantly smaller than the otherwise smallest cross-sectional area throughout the flow path. Accordingly, ‘C’ can be taken from the specification of orifice plate 322, with respect to air, or determined with a calibrating measurement. ‘P_{EXHALED}’ is the pressure in kilograms/(meter·second²) measured while subject 101 is blowing and ‘P_{AMBIENT}’ is the ambient atmospheric pressure and, effectively, the pressure downstream of orifice plate 322. P_{AMBIENT} is measureable with sensor 321 when the subject is not blowing, or with a separate pressure sensor (not shown) outside of test chamber 314. The specific heat ratio ‘k’ is a property of the air (also dimensionless), which for a significant accuracy over a useful range of humidity is 1.40. Over the temperature range and pressures compatible with human life, the gas compressibility factor ‘Z’ (dimensionless) is effectively 1.000 (where air, even humid air, behaves sufficiently like an ideal gas). ‘M_{EXHALED}’ is the molar mass of the exhaled air (kilograms/mole), which is lower as the humidity increases, yet higher as the level of CO₂ increases, forming at least a partial offset of errors were the value for M_{AMBIENT} to be used instead of a more closed measured, calculated, or estimated value. ‘R’ is the universal gas law constant=8.3145 joules/(mol·K).

During an exhalation (or inhalation), measurements suitable for calculating ΔV_{EXHALED} from EQ. 1 may be acquired many times per second (e.g., 100), and each computed value multiplied by the interval (in seconds) since the last reading (as measured by the clock), and the resulting incremental volume (in meters³) accumulated (effectively integrating the gas flow rate).

Humidity sensor 323 is an absolute humidity sensor. An example of such a sensor is based on thermal conductivity and measures the ability of the surrounding air to absorb heat, a property that varies with the absolute humidity (AH), i.e., mass of water per volume of air. Given an inhalant 111 characterized by a sensor 323 measuring absolute humidity (AH_{INHALED}, not shown in FIG. 1) and volume V_{INHALED}, the mass of water m_{Vinhaled} contained in the inhalant 111, is simply the product of the volume and the absolute humidity.

As discussed above in conjunction with FIG. 2 and relative humidity sensor 223, relative humidity (RH) can be converted to absolute humidity (AH) using the following equations:

$$RH = 100 \times \frac{{P}_{V}}{{P}_{W} ( T )} \qquad EQ . 2$$

Where ‘P_{V}’ is the actual vapor pressure of water and ‘P_{W}’ is the vapor pressure if saturated, which is dependent on temperature (T). [Note that in the field, P_{W} is usually called out as ‘e_{W}’, but herein, P_{W} is the variable to avoid confusion with ‘e’, the base of the natural logarithm, in the equations that follow.] The source for EQ. 2 is the definition of relative humidity.

$${P}_{W} ( T ) = 6.112 \times {e}^{\frac{17.62 ( T - 273.15 )}{T - 30.03}} \qquad EQ . 3$$

Where ‘T’ is absolute temperature in degrees Kelvin. EQ. 3 is accurate to within 0.1% for atmospheric pressures from sea level to the peak of Everest, even without an enhancement factor (not included in EQ. 3) sometimes used to correct for the departure of moist air from the behavior of an ideal gas. The source for EQ. 3 is the “Guide to Meteorological Instruments and Methods of Observation”, World Meteorological Organization, Geneva, Switzerland, 2008, modified to use absolute temperature, from the original using degrees Centigrade.

Absolute humidity is defined as:

$$AH = \frac{{m}_{V}}{V} \qquad EQ . 4$$

Where ‘m_{V}’ is the mass of the water vapor in the volume ‘V’, which is also the density of water vapor.

The Ideal Gas Law (PV=nRT) is sometimes written in the molar form:

P_{V}V=m_{V}R_{W}T EQ. 5:

Where, for water vapor, ‘P_{V}’, ‘V’, and ‘T’ are the actual vapor pressure of water (in Pascals, kg*m⁻¹*s⁻²), volume (in cubic meters, m³), and temperature (in Kelvin, K), respectively, and where ‘m_{V}’ is the mass of water vapor (in grams), ‘R_{W}’ is the specific gas constant for water vapor, 461.5 (in J*kg⁻¹*K⁻¹), which is the universal gas constant divided by the molar mass of water, about 18.02 grams/mole.

Solving EQ. 5 for m_{V}/V and substituting with EQS. 2 & 4 gives:

$$\frac{{m}_{V}}{V} = \frac{{P}_{V}}{{R}_{W} T} = AH = RH \times \frac{{P}_{W} ( T )}{100 \times {R}_{W} T} \qquad EQ . 6$$

Substituting from EQ. 3:

$$\frac{{m}_{V}}{V} = AH = RH \times \frac{6.112}{100 \times {R}_{W} T} \times {e}^{\frac{17.62 ( T - 273.15 )}{T - 30.03}} \qquad EQ . 7$$

Whereby the density of water vapor in air can be determined from either the absolute humidity (AH) or the relative humidity (RH) and temperature (T). Multiplying through by a volume V (for example, of exhalant), gives the mass of water vapor in the volume (for example, in the exhalant):

$${m}_{V} = AH \times V = RH \times V \times \frac{6.112}{100 \times {R}_{W} T} \times {e}^{\frac{17.62 ( T - 273.15 )}{T - 30.03}} \qquad EQ . 8$$

However, it is commonly the case that air inhaled is not dry, so the water lost in respiration is only the incremental water mass added in respiration:

water_loss=m_{V}_{_{EXHALED}}−m_{V}_{_{INHALED}} EQ. 9:

Where water_loss is in grams, but because the density of water throughout the range of drinkable temperatures is 1 g/ml, this equates to milliliters of water.

Thus, by measuring or estimating the volume of exhalation, accumulated over a number of breaths in an interval, the mass of water loss during that interval can be determined.

Thus, if a sensor is read in absolute humidity, the amount (volume) “V_{R}” of replacement water to drink, in liters, is (from EQ. 8 and 9):

V_{R}=AH_{EXHALED}V_{EXHALED}−AH_{INHALED}V_{INHALED} EQ. 10:

Or, for a sensor read in relative humidity, this becomes:

$${V}_{R} = {RH}_{EXHALED} {V}_{EXHALED} \times \frac{6.112}{100 \times {R}_{W} {T}_{EXHALED}} \times {e}^{\frac{17.62 ( {T}_{EXHALED} - 273.15 )}{{T}_{EXHALED} - 30.03}} - {RH}_{INHALED} {V}_{INHALED} \times \frac{6.112}{100 \times {R}_{W} {T}_{INHALED}} \times {e}^{\frac{17.62 ( {T}_{INHALED} - 273.15 )}{{T}_{INHALED} - 30.03}} \qquad EQ . 11$$

Where V_{R} in grams corresponds to milliliters of replacement water to drink to replace water loss from respiration.

An incremental volume for replacement water to drink, ∂V_{R} may be determined and accumulated for each breath during an interval. The number of breaths B in the interval can be estimated (e.g., from a respiratory rate times the duration of the interval) or actually counted during the interval. ∂V_{R} is also dependent on the volume of each breath, which can be measured and/or estimated.

In some embodiments, both the inhaled and exhaled volumes of EQS. 10 and 11 can be individually measured, and this is the preferred operation. However, in an alternative embodiment, a reasonable estimation is that V_{INHALED}=V_{EXHALED} whereby only one need be measured or estimated, however this introduces a minor error, insofar as the volume of air inhaled is not necessarily the volume exhaled for at least two reasons: First, the amount of water vapor differs between the inhalant and exhalant; and second, the temperatures of the inhalant and exhalant usually differ. The moles of carbon dioxide produced by cellular metabolism and removed in the exhalant are nearly a one-for-one replacement for the oxygen present in the inhalant, but absent from the exhalant, at least when subject 101 is in a steady state (e.g., resting after an interval of resting, or exercising after an interval of exercise), however this need not be the case when the amount of exercise has recently changed.

For some embodiments, an acceptable approximation can be that during sustained heavy exercise, a relatively constant volume is inhaled/exhaled each breath, so an actual measurement during one brief interval of heavy exercise could be used as a proxy to estimate breathing volume during a different extended interval of heavy exercise. The intervals may even come from different days (e.g., volume might be measured once per week). In one embodiment, a separate measurement of breath volume may be made during light or moderate exercise, e.g., rest and/or walking, and used as an estimate of breathing volume for intervals of comparable exertion, for example as detected by an activity sensor or heart rate measurement. Thus, a collection of breath volumes, personalized to an individual, could be accumulated for each of at least one context and stored in the subject profile data. Subsequently, when a similar context arises, a breath volume previously measured can be selected on the basis of which context is most similar, or if a plurality of contexts are sufficiently similar, the corresponding breath volume measurements can be combined, e.g., with a weighted average, to estimate the current breath volume.

In some embodiments, the relative humidity of the exhalant can be considered to be 100%, as the lungs are a terrifically humid place. For such embodiments, only the ambient humidity, or inhalant humidity, needs to be determined. In some of such embodiments, where the reasonable estimation that V_{INHALED}=V_{EXHALED} is used, only V_{INHALED} need be measured, in which case, mouth port 212, 312 may comprise a valve (not shown) to limit air-flow through the test chamber to be only inhaled air. Note that in such embodiments, blood cell sensor 224, 324 would not be used.

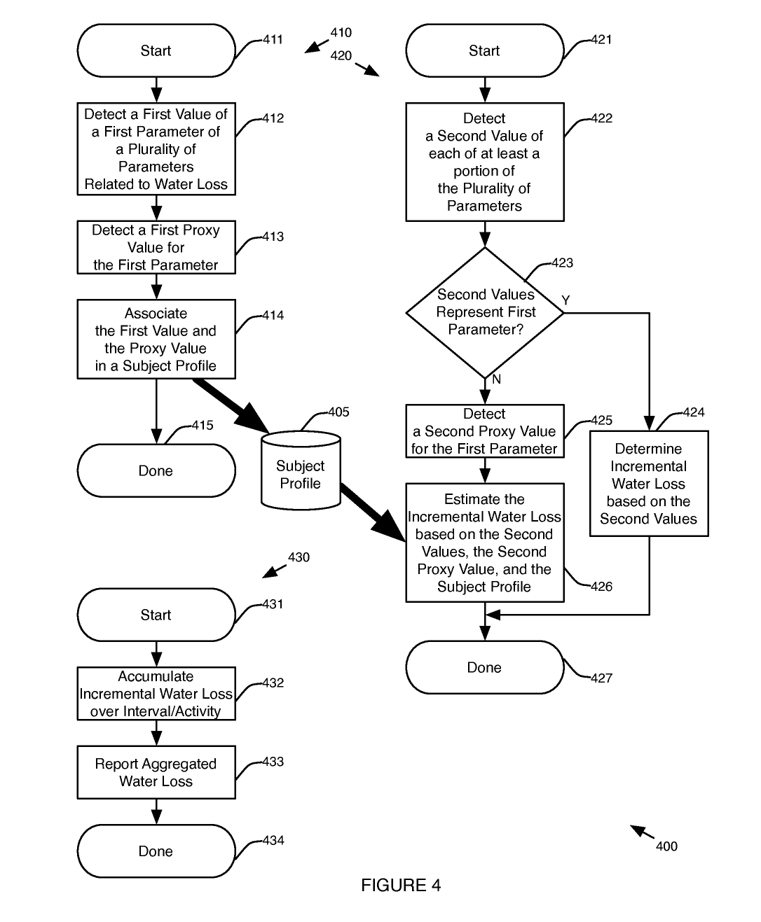

FIG. 4 is a flowchart showing one example respiration water loss monitoring process 400, comprising three portions: Water loss profile adjustment process 410, water loss determination process 420, and water loss reporting process 430.

Water loss profile adjustment process 410 is performed one or more times, beginning at step 411 with the subject associated with the profile is predetermined.

At step 412, a first value of a first parameter related to water loss is detected. This first parameter is one of a plurality of parameters directly related to water loss. The plurality of parameters may include (without limitation) the absolute humidity of inhalant or ambient air, the absolute humidity of exhalant, the relative humidity of inhalant or ambient air, the relative humidity of exhalant, the temperature of inhalant or ambient air, the temperature of exhalant, the volume of inhalant, the volume of exhalant, and the subject's breathing rate (or breathing interval). The first value is measured using one or more sensors of the water loss monitor (e.g., example water loss monitor 200 or 300).

At step 413, a first proxy value for the first parameter is detected. For each of at least some of the plurality of parameters directly related to water loss, including the first parameter from step 412, there is at least one proxy value that can be detected through the sensors of the water loss monitor. Such proxy values are usable to estimate the first parameter. In some cases, a proxy value may be a tuple of component values detected through the sensors of the water loss monitor. For example, one or more of heart rate, activity level, or particular recognized exercise may be used as a proxy value for breathing rate. In one embodiment, heart rate might be considered alone as the proxy for breathing rate. The first value detected might be a breathing rate of 25 breaths per minute and the first proxy value detected as a heart rate of 100 beats per minute. In another embodiment, heart rate and recognized exercise might be considered together as the proxy for breathing rate, in which case the proxy value would be a heart rate of 100 beats per minute while walking.

At step 414, the first value and first proxy value (whether an individual component value or a tuple consisting of multiple component values) are associated in a subject profile 405. The process 410 concludes at step 415.

Over multiple iterations of water loss profile adjustment process 410, multiple first values will have been accumulated and associated with corresponding first proxy values. Over longer periods of time, e.g., months, older associations in subject profile 405 are given less weight, and in some embodiments may be forgotten completely. In shorter periods of time, within the same day or over a few days or weeks, such information may be analyzed and consolidated (a step not shown). For example, a line or other equation could be fitted to relate the multiple first values to the associated first proxy values, as might be done using a least squares fit. In an embodiment where heart rate is used as a proxy for breathing rate, a line equation “y=ax+b” might be fitted, where ‘y’ is breathing rate in breaths per minute, ‘x’ is heart rate in beats per minute, and ‘a’ and ‘b’ are constants determined by the least squares fitting after two or more iterations of water loss profile adjustment process 410 has taken place. In such embodiments, the constants (e.g., ‘a’ and ‘b’) so determined can also be stored in subject profile 405.

As another example, an embodiment might provide that the rate of perspiration together with the ambient temperature and ambient relative humidity is a proxy for absolute humidity of the subject's exhalant. A more complex proxy, that is, one with a plurality of component values (vs. an individual component value), can require a more complex equation when fitted to summarize the multiple entries into the subject profile 405. However, this is not always the case: If recognized exercise is used as proxy component, e.g., if controller 220 can distinguish between standing, walking, cycling, and running on the basis of measurements taken with motion sensor 226, then the recognized exercise, as a component of the proxy, can be used to select between each of four separately fitted equations, one for each of standing, walking, cycling, and running.

In some embodiments, some of the plurality of parameters directly related to water loss may not need proxy values. For example, a measurement of ambient temperature or ambient humidity might be usable over an entire exercise interval. For example, if exercise is always conducted in a gym, the ambient temperature and humidity may be controlled such that change over even a protracted workout might be negligible.

Water loss determination process 420 starts at step 421, where water loss profile adjustment process 410 has been performed at least one time. At step 422, a second value of each of at least a portion of the plurality of parameters is detected.

At step 423, a determination is made as to whether the first parameter is among the second values. If so, process 420 continues at step 424 and an incremental water loss amount is determined on the basis of the second values. Otherwise, at step 425, a second proxy value for the first parameter is detected, and at step 426, the incremental water loss amount is estimated on the basis of the second values, the second proxy value, and the subject profile. The estimation of incremental water loss takes advantage of the association between at least one first value of the first parameter, and the first proxy value corresponding thereto. Steps 425-426 provide the advantage that when some parameter cannot be directly determined, e.g., due to a particular sensor reading being not available or the detected value being otherwise inadequate (e.g., the reading is a nonsensical value).

Note that the plurality of parameters directly related to water loss may comprise those relating to water loss through perspiration. A portion of the incremental water loss determined or estimated in steps 424 or 426, respectively, may be for water lost by perspiration.

Water loss reporting process 430 starts at step 431, based on an inciting event. Such an event may be a user interaction, e.g., pressing a user interface control, such as button 229, to indicate the start of an exercise interval or that a previously recommended amount of water has been consumed, so a new interval of water loss should be initiated. Alternatively, the event may be the initiation of increased activity detected with a motion sensor (e.g., 226, 336) or a recognized pattern of motion. Depending upon the embodiment, any previously aggregated water loss may or may not be 1) reset to zero; and, 2) separately stored for later reporting. Also, the occurrence of the inciting event may be noted in the memory.

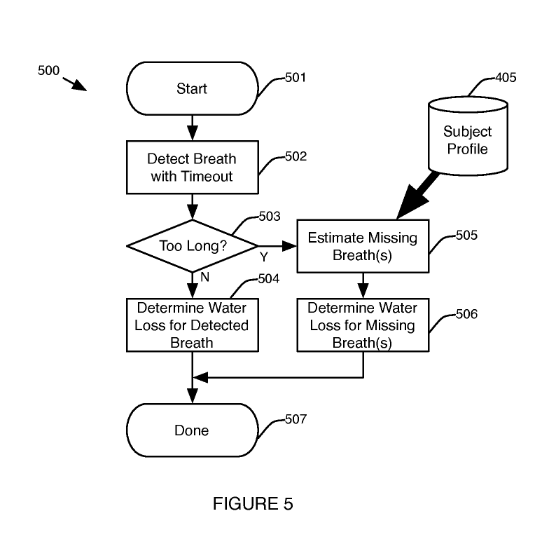

At step 432, the incremental amount of water loss (e.g., as determined with process 420) is accumulated over the interval or activity that initiated process 430. In one embodiment, incremental water loss is determined with each breath. In other embodiments, the incremental water loss is determined for at least one breath, and an aggregate value determined based on at least that value and an estimated number of breaths, as described below in conjunction with FIG. 5. In some embodiments, a portion of incremental water loss due to respiration may be determined on the basis of each breath, while another portion due to perspiration may be determined on a different basis (e.g., on a per minute basis):

The reporting of the amount of aggregated water loss from step 432 may be continuous (e.g., updating whenever more incremental water loss is accumulated), or the aggregated water loss can be reported at the end of the interval, at the end of the detected exercise, or when a particular volume of water loss is detected. For example, the report of step 433 might be a notice given whenever the accumulated water loss amounts to another half-glass to be replaced. When the interval is ended, process 430 ends at step 434. In some embodiments, reporting the amount of water loss can be achieved by storing the accumulated water loss in memory for later review (not shown), or, for example, by uploading the amount to a web site (connection not shown) for later review or for sharing with friends, a coach, or a doctor (not shown).

FIG. 5 is a flowchart showing one example breathing rate estimation process 500 for use when detection of each individual breath is either inconvenient or unreliable. Breathing rate estimation process 500 is suitable for use to monitor water loss, particularly through respiration, as discussed above. Breathing rate estimation process 500 begins at step 501, where water loss profile adjustment process 410 has been performed at least one time. At step 502, the subject's breath is detected but with a timeout, a maximum amount of time after which, at least one breath is estimated to have been taken, but for whatever reason was not measured. Typically, this is because the subject is not exhaling through the test chamber 214 or 314, as a matter of comfort or convenience.

At step 503, a test is made to determine whether a breath was timely detected in step 502. If so, then at step 504 the incremental water loss for the detected breath is determined, e.g., by the process 420. Otherwise, at step 505, a number of missed breaths is estimated. In an embodiment where the first parameter in water loss profile adjustment process 410 is breathing rate (or breath interval), and at least one first value of the first parameter and associated first proxy value are stored in subject profile 405, then the missing breaths are estimated based on a second proxy value and the subject profile, after which, at step 506, the water loss for the missing breaths is determined (e.g., by process 420). Regardless of the steps taken to determine the incremental water loss for the missing breath(s), breath rate estimation process 500 ends at step 507, but may be immediately restarted to detect for the subsequent breath(s).

FIG. 6 illustrates an example airborne blood cell sensor 600, suitable for use in various embodiments of the present invention, e.g., as blood cell sensors 224, 324 in FIGS. 2 and 3. Blood cell sensor 600 comprises a hollow body 601 with optically reflective elements (e.g., 604, 606) along its length, on opposite walls. At least a portion 610 of the exhalant 121 passes through the interior of hollow body 601, the portion shown while inside sensor 600 as exhalant flow 611 and when exiting as flow 612.

Emitter 602, which can comprise one or more LEDs or laser diodes, emits at least one beam 603 toward first reflective element 604. The beam reflects of the first reflective element 604 as beam 605, directed toward the second reflective element 606, and so on until the multiply-reflected beam reaches optical sensor 620. The initial beam 603 and the many reflected beams (e.g., 605) cross the exhalant flow 611 repeatedly. Accordingly, if there are particles able to absorb certain wavelengths of light, then these particles will have many opportunities to absorb such wavelengths, if present in the at least one beam 603, before detection by optical sensor 620.

Hemoglobin, when oxygenated as would be expected while exposed to air, is noted to have two particularly strong absorption bands at 541 nm (a greenish-cyan color) and 577 nm (a greenish-yellow color). Other wavelengths are considerably less strongly absorbed by hemoglobin. Provided that emitter 602 produces a first at least one beam 603 that is either 541 nm or 577 nm, sensor 620 can detect a more pronounced dip during exhalation phase 120 when blood cells are present in exhalant flow 611. Other wavelengths are less strongly absorbed by hemoglobin, e.g., yellow at 595 nm and longer wavelengths in the orange and red bands, are absorbed only about 1/15th as much, or less. Accordingly, a reference beam of such a less-absorbed color emitted as a second one of the at least one beam 603 can be used as an absorption reference, for example to mitigate false positive readings induced by a buildup of condensation of the breath on the mirrors such as 604, 606 or on sensor 620. Thus, if during exhalation phase 120, the second beam 603 can be used as a reference, while the first beam 603 can be used to detect for the presence of oxygenated hemoglobin, indicating the presence of blood cells in the exhalant flow 611. In some embodiments, the exhalant portion 610 may be further constrained to pass through a replaceable sleeve 630, comprising a material transparent at least to the optical wavelengths being monitored. This helps to keep mirrors (e.g., 604, 606) clean, but provides a surface (the inner surface of sleeve 630) where deposition of blood cells (if any) from exhalant can accumulate to provide an increasing detection signal as the deposition builds up over multiple breaths. An empirical calibration of airborne blood cell sensor 600 can be predetermined, and nulled when a new sleeve 630 is inserted. Thereafter, if and when enough attenuation is detected in a hemoglobin wavelength (e.g., 541 nm or 577 nm), a warning is be provided, particularly if the same level of attenuation is not observed in another “non-hemoglobin” wavelength (e.g., 595 nm or longer).

The foregoing describes a system and method for monitoring at least the exhalant of a subject to determine and report water loss and other detected exhalant components.

We claim:

1. A system for monitoring water loss comprising:
at least one humidity sensor;
an air flow sensor inside a test chamber, the test chamber having a mouth port and an ambient port;
at least one other sensor that provides a proxy value related to one of breathing rate of a subject and breath volume of the subject;
a memory; and,
a controller having communication with said at least one humidity sensor, the air flow sensor, said at least one other sensor, and memory;
wherein the controller is configured to:
determine a first humidity of an environment with said at least one humidity sensor,
determine a first breath volume of one of an inhalation and an exhalation based on readings from the air flow sensor as the subject breathes through the mouth port,
determine a first breathing rate based on readings from at least one of the air flow sensor and at least one other sensor,
determine a first proxy value based on readings from said at least one other sensor,
store in a profile in the memory, an association of the first proxy value with at least one of the first breath volume and the first breathing rate,
determine a plurality of second proxy values based on readings from said at least one other sensor over a interval,
determine an amount of water loss for the interval based on at least the first humidity, the plurality of second proxy values, and the profile,
the controller further having communication with a display, wherein the amount of water loss is reported on the display.

2. The system of claim 1 wherein said at least one other sensor comprises a motion sensor.

3. The system of claim 2 wherein said first and second proxy values each comprises an activeness reading.

4. The system of claim 3 wherein the interval is determined by the activeness readings.

5. The system of claim 2 wherein said first and second proxy values each comprises a recognized activity.

6. The system of claim 5 wherein the interval is determined by the recognized activity.

7. The system of claim 1 wherein said at least one other sensor comprises a heart rate monitor.

8. The system of claim 7 wherein the first and second proxy values each comprises heart rate.

9. The system of claim 7 wherein said heart rate monitor comprises an optical pulse oximeter.

10. The system of claim 9 wherein the first breathing rate is determined with the optical pulse oximeter.

11. The system of claim 1 wherein said at least one other sensor comprises a perspiration sensor and the first and second proxy values each comprise a measured perspiration.

12. The system of claim 11 wherein the amount of water loss is further based on the measured perspiration.

13. The system of claim 1 further comprising a perspiration sensor and the amount of water loss is further based on a measured perspiration.

14. The system of claim 1 wherein a first humidity sensor of said at least one humidity senor is inside the test chamber and the controller is further configured to:
determine a second humidity of an exhalation of a subject with the first humidity sensor, and
wherein the amount of water loss is further determined based on the second humidity.

15. The system of claim 1 wherein a first humidity sensor of said at least one humidity sensor senses absolute humidity.

16. The system of claim 1 wherein a first humidity sensor of said at least one humidity sensor senses relative humidity, the system further comprising:
a temperature sensor in proximity the first humidity sensor, the controller having communication with the temperature sensor;
wherein, the first humidity is further determined with the temperature sensor.

17. A method for monitoring water loss comprising the steps of:
accepting, by a controller, a first humidity of an environment;
reading, by the controller from a memory, a profile comprising an association of a first proxy value with at least one of a first breath volume and a first breathing rate;
determining, by the controller, a plurality of second proxy values for a subject based on readings over an interval from at least one sensor, the at least one sensor comprising at least one of a heart rate monitor, a perspiration sensor, and a motion sensor;
determining, by the controller, an amount of water loss by the subject for the interval based on at least the first humidity, the plurality of second proxy values, and the profile; and,
reporting, by the controller the amount of water loss.

18. The method of claim 17 wherein the at least one sensor comprises a motion sensor, and the plurality of second proxy values each comprise at least one of an activeness reading and a recognized activity, the method further comprising the step of:
determining the interval, by the controller, based on the at least one of the activeness reading and the recognized activity.

19. The method of claim 17 wherein the amount of water loss is further based on readings during the interval by the controller of a perspiration sensor.

20. The method of claim 17 further comprising the step of:
accepting, by the controller, a second humidity of air exhaled by the subjection through a test chamber comprising a humidity sensor readable by the controller;
wherein the amount of water loss is further based on the second humidity.
